(19) United States
(12) Patent Application Publication
(10) Pub. No.: US 2010/0167290 A1
(43) Pub. Date: Jul. 1, 2010

(54) MOLECULE ATTACHMENT TO NANOPARTICLES

(76) Inventors: Robert Elghanian, Wilmette, IL (US); Chad A. Mirkin, Wilmette, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(21) Appl. No.: 12/527,623

(22) PCT Filed: Feb. 27, 2008
(86) PCT No.: PCT/US08/55133

§ 371 Date: Aug. 21, 2009

Related U.S. Application Data
(60) Provisional application No. 60/903,728, filed on Feb. 27, 2007.

(51) Int. Cl. C12Q 1/68; C07H 21/00; C07K 14/00; C07K 9/00; C07K 16/00; G01N 33/553

(52) U.S. Cl. 435/6; 536/23.4; 530/350; 530/322; 530/391.1; 436/525; 977/773; 977/904

(57) ABSTRACT

Disclosed herein are molecule-modified nanoparticles and methods of making and using the same. More specifically, disclosed herein are molecule-modified nanoparticles wherein the molecule is attached to the surface of the nanoparticle via an oligonucleotide. Also disclosed are methods of preparing nanoparticles having oligonucleotides and molecules (e.g., biomolecules, such as proteins, peptides, antibodies, lipids, and/or carbohydrates) attached to the nanoparticle surface, wherein the oligonucleotide and molecule are covalently attached. Further disclosed are methods of detecting an analyte of interest using these disclosed molecule-modified nanoparticles.

### CROSS-REFERENCE TO RELATED APPLICATIONS

This application claims priority to U.S. Provisional Application No. 60/903,728, filed Feb. 27, 2007, which is incorporated herein by reference in its entirety.

### STATEMENT OF GOVERNMENT INTEREST

This invention was made with government support under Air Force Office of Scientific Research (AFOSR) grant No. FA9550-05-1-0348. The government has certain rights in this invention.

### BACKGROUND

In recent years, significant progress has been made toward the design and synthesis of nanostructures suitable for biological applications. The ability to assemble nanomaterials with precise control over size and morphology is largely dependent on the availability of well defined, macromolecular building blocks. The capacity to attach further moieties to a nanostructure has been limited by lack of control of attachment, limited stability of the resulting nanostructures, and undesirable aggregation and precipitation of nanostructures during formation. Thus, a need exists to provide methods of preparing nanostructures having biological moieties appended such that the nanostructure is modified in a controlled fashion, is stable, and does not result in much, if any, aggregation.

### SUMMARY

In light of the foregoing, the present invention provides nanoparticles having biological moieties appended such that the nanostructure is modified in a controlled fashion, is stable, and does not result in much, if any, aggregation. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

Thus, in one aspect, disclosed herein is a molecule-modified nanoparticle, comprising a molecule covalently attached to an oligonucleotide, the oligonucleotide further covalently attached to the surface of the nanoparticle. In various embodiments, the molecule is attached at a first end of the oligonucleotide and the nanoparticle is attached at a second end of the oligonucleotide. In some embodiments, the molecule is a biomolecule, and can be a protein, peptide, antibody, lipid, carbohydrate, or a combination thereof. In a specific embodiment, the molecule is an antibody. In various embodiments, the nanoparticle is metallic. In specific embodiments, the metal is gold. In a specific embodiment, the nanoparticle is gold and the oligonucleotide is attached to the surface of the nanoparticle via a linkage comprising a sulfur atom. In some embodiments wherein the nanoparticle is gold, the gold nanoparticle is about 10 nm to about 100 nm. In some embodiments, the oligonucleotide has 20 to 150 nucleobases.

In another aspect, disclosed herein is a method of preparing a molecule-modified nanoparticle as disclosed herein comprising contacting a nanoparticle with an oligonucleotide having a functional group at one distinct location and a leaving group a second distinct location to form an oligonucleotide-modified nanoparticle such that the oligonucleotide is attached to a surface of the nanoparticle via the functional group; and contacting the resulting oligonucleotide-modified nanoparticle with a molecule having a nucleophile under conditions sufficient to permit displacement of the leaving group on the oligonucleotide by the nucleophile of the molecule to form the molecule-modified nanoparticle.

In still another aspect, disclosed herein are methods of detecting an analyte in a sample using a molecule-modified nanoparticle comprising contacting the sample with a molecule-modified nanoparticle as disclosed herein under conditions to permit binding of the analyte to the molecule and detecting the resulting nanoparticle-bound analyte, wherein the binding of the analyte to the molecule-modified nanoparticle produces a detection event. In some embodiments, the detection event comprises a change in color, a change in the ability of the molecule-modified nanoparticle to conduct electricity; a change in fluorescence, a change in solubility to produce a precipitate, a change in the scattering of light; or change in melting temperature of a probe oligonucleotide hybridized to the oligonucleotide of the molecule-modified nanoparticle. In other embodiments, the concentration of the analyte in the sample can be calculated. In specific embodiments, the methods of detecting disclosed herein are sufficiently sensitive to detect an analyte at a concentration of about 300 fM (femptomolar).

Illustrating certain non-limiting benefits and utilities of the present invention, such a nanoparticle can be contacted with a cancer cell expressing one or more antigens, with one or more of the aforementioned hydrophilic moieties conjugated with one or more antibodies against such antigen(s).

### BRIEF DESCRIPTION OF THE DRAWINGS

FIG. 1 shows a scheme for prior methods of preparing an antibody-modified nanoparticle, where the antibody and oligonucleotide are separately attached to a nanoparticle surface.

FIG. 2 shows a scheme for a method as disclosed herein for preparing a molecule-modified nanoparticle, where the molecule is attached to the nanoparticle via an oligonucleotide.

FIG. 3 shows a calibration of signal across a range of analyte (here, Prostate Specific Antigen (PSA)) concentrations.

FIG. 4 shows detection of PSA as background noise in various serum samples, using the methods disclosed herein.

FIG. 5 shows a calibration curve using 30% human serum spiked with varying concentrations of a PSA standard, in the presence (left bar) or absence (right bar) of a probe for the presence of PSA.

FIG. 6 in Panel A shows binding assay results of the detection assays disclosed herein in the presence of varying concentrations of the molecule-modified nanoparticle to show the binding mode of the molecule-modified nanoparticle through the oligonucleotide probes hybridized to the oligonucleotides of the molecule-modified nanoparticle and on the surface of the slide, as a means of detecting the presence of the oligonucleotide probe; In Panel B is shown a surface bound antigen (here PSA) on the surface of a slide in the presence of various conditions to show the specificity of the molecule-modified nanoparticle toward detection of the target analyte of the molecule of the molecule-modified nanoparticle (here an antigen for PSA)—well 1 having the bio barcode probe plus excess antibody; well 2 having the bio barcode probe plus excess antigen; well 3 having the bio barcode plus assay buffer; and well 4 having the bio barcode probe.

### DETAILED DESCRIPTION

Disclosed herein are molecule-modified nanoparticles, wherein the nanoparticles have molecules attached to at least a portion of their surfaces via oligonucleotides. Further disclosed are methods of preparing the same. In comparison to prior known methods of molecule attachment to nanoparticles, the disclosed methods allow for better control over the loading of the molecule to the nanoparticle, and/or result in molecule-modified nanoparticles which are more stable and have less aggregation.

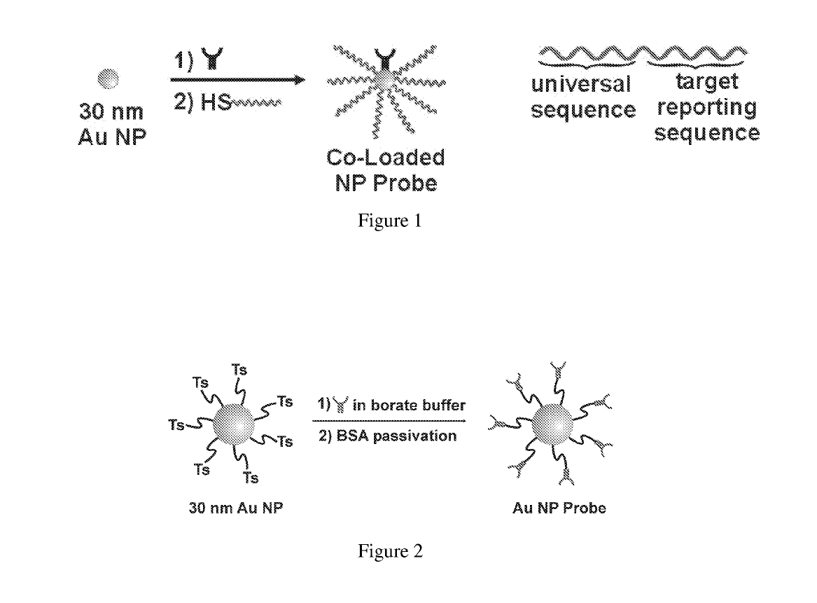

Prior means of preparing nanoparticles having both oligonucleotides and molecules attached were prepared by first conjugating the molecule (e.g., a biomolecule, such as an antibody) to a nanoparticle surface followed by addition of oligonucleotides on the remainder of surface where the surface voids were filled by oligonucleotides. The procedures used in the past have often been difficult to control, and large amounts of precipitated nanoparticles were generally observed during the isolation of nanoparticles. The modified nanoparticles prepared in this manner also appeared to have a limited shelf-life, and thus, long term usage involved daily preparation of the probes. This prior method is depicted in FIG. 1.

The methods disclosed herein use oligonucleotides which have a functional group at one distinct location and a leaving group at a second distinct location. The oligonucleotides are first loaded onto the nanoparticle through the functional group to form an oligonucleotide-modified nanoparticle, and the resulting oligonucleotide-modified nanoparticle can be isolated and stored until needed. The oligonucleotide-modified nanoparticle can then be further modified through the leaving group on the oligonucleotide with a molecule (e.g., a biomolecule, such as a protein, a peptide, an antibody, a lipid, or a carbohydrate). The oligonucleotide is capable of reacting with the surface of a nanoparticle via a functional group at one end, e.g., through disulfide conjugation, and also with a nucleophile on the molecule via the leaving group on the opposite end of the oligonucleotide. The disclosed method is outlined in FIG. 2, where Ts is tosyl.

Loading the oligonucleotide onto the nanoparticle before the molecule can maximize loading of the oligonucleotide. Increased or high density loading of the oligonucleotide allows for maximum amplification of a recognition signal in detection assays. Greater amplification allows for more sensitive detection of an analyte of interest, as the recognition signal is amplified to detect that analyte's presence. Additionally and alternatively, an increase in the number of oligonucleotides on the nanoparticle can be achieved by using a larger nanoparticle.

### Nanoparticles

In practice, methods are provided using any suitable nanoparticle which can be modified to have oligonucleotides attached thereto. The size, shape and chemical composition of the nanoparticles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of nanoparticles having different sizes, shapes and/or chemical compositions, as well as the use of nanoparticles having uniform sizes, shapes and chemical composition, are contemplated. Examples of suitable particles include, without limitation, aggregate particles, isotropic (such as spherical particles) and anisotropic particles (such as non-spherical rods, tetrahedral, prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); and Ushida et al., J. Phys. Chem., 95, 5382 (1992).

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of Nanoparticles Comprising Polymerized Methylmethacrylate (MMA) is Described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers). Suitable nanoparticles are also commercially available from, for example, Ted Pella, Inc. (gold), Amersham Corporation (gold) and Nanoprobes, Inc. (gold). Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Also, as described in U.S. Patent Publication No 2003/0147966, nanoparticles comprising materials described herein are available commercially, or they can be produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, Jan. 1990, 16-47. As further described in U.S. Patent Publication No 2003/0147966, nanoparticles contemplated are produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or amount surface area that can be derivatized as described herein.

### Oligonucleotides

As used herein, the term “oligonucleotide” refers to a single-stranded oligonucleotide having natural and/or unnatural nucleotides. Throughout this disclosure, nucleotides are alternatively referred to as nucleobases. The oligonucleotide can be a DNA oligonucleotide, an RNA oligonucleotide, or a modified form of either a DNA oligonucleotide or an RNA oligonucleotide.

Naturally occurring nucleobases include adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopu-rine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “unnatural” nucleobases include those described in U.S. Pat. No. 5,432,272 and Freier et al. Nucleic Acids Research, 25:4429-4443 (1997). The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808; in Sanghvi, Antisense Research and Application, Crookei and B. Lebleu, eds., CRC Press, 1993, Chapter 15; in Englisch et al., Angewandte Chemie, International Edition, 30:613-722 (1991); and in the Concise Encyclopedia of Polymer Science and Engineering, J. I. Kroschwitz Ed., John Wiley & Sons, 1990, pages 858-859, Cook, Anti-Cancer Drug Design, 6, 585-607 (1991), each of which are hereby incorporated by reference in their entirety. Nucleobase also includes compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. The oligonucleotide incorporated with the universal base analogues is able to function as a probe in hybridization, as a primer in PCR and DNA sequencing. Examples of universal bases include but are not limited to 5′-nitroindole-2′-deoxyriboside, 3-nitropyrrole, inosine and pypoxanthine.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

Modified oligonucleotides wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed oligonucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997).

Nanoparticles for use in the methods provided are modified with an oligonucleotide, or modified form thereof, which is from about 5 to about 150 nucleotides in length. Methods are also contemplated wherein the oligonucleotide is about 5 to about 140 nucleotides in length, about 5 to about 130 nucleotides in length, about 5 to about 120 nucleotides in length, about 5 to about 110 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

In still other aspects, oligonucleotides comprise from about 8 to about 80 nucleotides (i.e. from about 8 to about 80 linked nucleosides). One of ordinary skill in the art will appreciate that methods utilize compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide in length.

### Oligonucleotide Sequences and Hybridization

Each nanoparticle utilized in the methods provided has a plurality of oligonucleotides attached to it. As a result, each oligonucleotide-modified nanoparticle has the ability to hybridize to a second oligonucleotide-modified nanoparticle, and/or when present, a free oligonucleotide, having a sequence sufficiently complementary. In one aspect, methods are provided wherein each nanoparticle is modified with identical oligonucleotides, i.e., each oligonucleotide attached to the nanoparticle has the same length and the same sequence. In other aspects, each nanoparticle is modified with two or more oligonucleotides which are not identical, i.e., at least one of the attached oligonucleotides differ from at least one other attached oligonucleotide in that it has a different length and/or a different sequence.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In some aspects, the oligonucleotide attached to the nanoparticle is complementary to a probe oligonucleotide. In various aspects, the oligonucleotide which is 100% complementary to the probe oligonucleotide, i.e., a perfect match, while in other aspects, the oligonucleotide is at least (meaning greater than or equal to) about 95% complementary to the probe compound over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the probe compound over the length of the oligonucleotide.

A probe oligonucleotide is an oligonucleotide used in a detection assay to assist in the detection of a analyte of interest. The probe oligonucleotide can be used in an assay such as a bio barcode assay, discussed below. See, e.g., U.S. Pat. Nos. 6,361,944; 6,417,340; 6,495,324; 6,506,564; 6,582,921; 6,602,669; 6,610,491; 6,678,548; 6,677,122; 6682,895; 6,709,825; 6,720,147; 6,720,411; 6,750,016; 6,759,199; 6,767,702; 6,773,884; 6,777,186; 6,812,334; 6,818,753; 6,828,432; 6,827,979; 6,861,221; and 6,878,814.

### Oligonucleotide Attachment to Nanoparticle

The oligonucleotides disclosed herein are modified to incorporate a leaving group at one distinct location and a functional group at a second distinct location. In some embodiments, the leaving group is toward one end of the oligonucleotide and the functional group is at an opposite end of the oligonucleotide. In specific embodiments, the leaving group is at one terminus of the oligonucleotide and the functional group is at an opposite terminus. The leaving group and functional group moiety can be attached at any portion of the oligonucleotide capable of being modified to have a leaving group and/or a functional group moiety.

The oligonucleotide is bound to the nanoparticle via a functional group moiety. Examples of sites on the oligonucleotide capable of being modified include, but are not limited to, a hydroxyl, phosphate, or amine. In some embodiments, the oligonucleotide has an unnatural nucleobase which incorporates a leaving group and/or a functional group moiety for attachment to a nanoparticle surface. In various aspects, the functional group is a spacer. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoparticle are well known in the art. See Whitesides, Proceedings of the Robert A. Welch Foundation 39th Conference On Chemical Research Nanophase Chemistry, Houston, Tex., pages 109-121 (1995). See also, Mucic et al. Chem. Comm. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabaretal., Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49:410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lec et al., J. Phys. Chem., 92:2597 (1988) (rigid phosphates on metals).

In one embodiment, the oligonucleotide has a disulfide functionality toward one end. This functional group can be achieved using, e.g., a dithiol phosphoramidite nucleobase (e.g., such as DTPA sold by Glen Research, Sterling, Va., USA). Selection of DTPA as functional group of the oligonucleotide is preferred because a free thiol may react with the leaving group end of the oligonucleotide to form self-aggregates of the oligonucleotide. However, any combination of functionality capable of attaching to a nanoparticle surface and leaving group moiety is contemplated which is stable under the disclosed conditions and able to provide the molecule modified nanoparticles.

### Oligonucleotide Density

Method are provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm², at least 50 pmol/cm², or 50 pmol/cm² or more.

In one aspect, methods are provided wherein the packing density of the oligonucleotides on the surface of the nanoparticle is sufficient to result in cooperative behavior between nanoparticles and between polynucleotide strands on a single nanoparticle. In another aspect, the cooperative behavior between the nanoparticles increases the resistance of the oligonucleotide to degradation.

### Molecule Attachment to Oligonucleotides

The oligonucleotide disclosed herein is modified with a leaving group at a distinct location. A leaving group, as used herein, refers to a moiety which is readily susceptible to nucleophilic attack by a nucleophile. Typical leaving groups include, but are not limited to, tosyl, mesyl, trityl, substituted trityl, nitrophenyl, chlorophenyl, fluorenylmethoxy carbonyl, and succinimidyl The preferred leaving group is tosyl. Modification of a 3′ or 5′ end of an oligonucleotide to provide a leaving group functionality is well known in the art. See, e.g., WO 93/020242 for methods of modifying an oligonucleotide with a leaving group.

The molecule is attached to the nanoparticle via nucleophilic displacement of the leaving group on the oligonucleophile. Nucleophiles on the molecule can be, for example, an amine, a hydroxyl, a carboxylate, a thiol, or any other moiety capable of displacing a leaving group. Conditions sufficient to permit displacement of a leaving group by a nucleophile are easily determined by one of skill in the chemical arts.

In some embodiments, the molecule disclosed herein is a target molecule for an analyte of interest. Examples of target molecules include proteins, peptides, lipids, carbohydrates, and the like. More specific examples include antibodies for an antigen of interest, small molecule receptors of an enzyme of interest, enzymes of small molecule receptor of interest.

### Detection Assays

The disclosed molecule-modified nanoparticles can be used in detection assays, such as the bio barcode assay. See U.S. Pat. Nos. 7,323,309; 6,974,669; 6,750,016; 6,268,222; 5,512,439; 5,104,791; 4,672,040; and 4,177,253; U.S. Publication Nos. 2001/0031469; 2002/0146745; and 2004/0209376; and International Patent Publication No. WO 05/003394, each of which is incorporated herein by reference in its entirety. Other detection assays for which an immobilized molecule is of use are also contemplated. Non-limiting examples of such assays include immuno-PCR assays; enzyme-linked immunosorbent assays, Western blotting, indirect fluorescent antibody tests, change in solubility, change in absorbance, change in conductivity; and change in Raman or IR spectroscopy. (See e.g., Butler, J. Immunoassay, 21(2 & 3):165-209 (2000); Herbrink, et al., Tech. Diagn. Pathol. 2:1-19 (1992); and U.S. Pat. Nos. 5,635,602 and 5,665,539, each of which is incorporated herein by reference).

The binding of the analyte to the molecule-modified nanoparticle will produce a change that can be detected, termed a “detection event.” Depending upon the assay being employed, that detection event can be a change in fluorescence (e.g., in embodiments where a fluorescent label used); a change in absorbance, a change in Raman spectroscopy; a change in electrical properties (e.g., increase or decrease in ability of sample or molecule-modified nanoparticle to conduct electricity); a change in light scattering; a change in solubility (e.g., analyte binding to the molecule-modified nanoparticle causes it to participate out of the assay solution), or some other change in physical or chemical properties that can be detected using known means.

Analytes can be detected at very low concentrations using the disclosed methods. In some embodiments, the analyte is present at a concentration as low as 300 fM. In various embodiments, the concentration of the analyte can be determined by comparing the detection event, e.g., change in absorbance or the like, and comparing that result to a calibration curve.

Additional aspects and details of the invention will be apparent from the following examples, which are intended to be illustrative rather than limiting.

### EXAMPLES

### Example 1

### Preparation of Tosylated Oligonucleotides

Oligonucleotides were prepared via standard phosphoramidite synthesis using Ultramild reagents from Glen Research on 1 μmmole scale. For 3′ dithiol functionalization and attachment of the oligos to the gold a DTPA monomer (Glen Research) was introduced at the 3′ end using either an A or G, CPG Ultramild support. 5′ Tosyl modification was introduced using a 5′ Tosyl T-phosphoramidite (Herrlein, et al., J. Am. Chem. Soc. 117:10151-10152 (1995)). The protected oligonucleotide was then deprotected in concentrated ammonium hydroxide at 55° C. for 15 minutes followed by 1.5 hours of standing at room temperature. Ammonium hydroxide was removed under a stream of nitrogen. The crude product was then purified by HPLC (0.03M triethylammonium acetate, 95% CH₃CN/5% 0.03M triethylammonium acetate) using a 1%/minute gradient at a flow rate of 3 mL/minute on a reverse phase column.

### Example 2

### Preparation of Tosyl-Oligonucleotide Nanoparticles

Tosyl-oligonucletide nanoparticles were prepared by addition of 1 O.D. of the tosylated oligonucleotide of Example 1 to 1 mL of 30 nm gold particles. The mixture was allowed to stand at room temperature for 24 hour. Following this initial incubation period, 10% sodium dodecyl sulfate (SDS) was introduced at a final concentration of 0.1% followed by addition of sodium chloride to a final concentration of 0.1M using a 1M salt solution. The mixture was then allowed to stand at room temperature for 48 hours. The conjugates were then harvested by centrifugation at 6800 rpm for 15 minutes using an eppendorf bench top centrifuge, washed twice with Nanopure water and finally suspended in Nanopure water and refrigerated.

### Example 3

### Preparing Molecule-Modified Nanoparticles

The molecule-modified nanoparticles were prepared by concentration of 3.0 mL of the tosyl-oligonucleotide nanoparticles of Example 2 down to 60 μL, by centrifugation and removal of the supernatant. To this concentrate was added 20 μL of a 0.2% Tween20 solution followed by 10 μg of a desired molecule in 20 μL PBS buffer pH 7.4.

In a specific example, PSA detection was desired, so polyclonal antibody from R&D Systems, anti-h Kallikrein-3 affinity purified goat IgG was used. To this mixture was added 100 μL of a 0.2M borate buffer solution at pH 9.5. The mixture was allowed to react at 37° C. for 24 hours at 550 rpm on an eppendorf Thermomixer R. To this mixture was added 10 μL of a 10% BSA solution and allowed to react for an additional 24 hours under the previous conditions. The molecule-modified nanoparticles were harvested by centrifugation at 5800 rpm for 15 minutes followed by washes using a pH 7.4 PBS buffer containing 0.1% BSA, 0.025% Tween20 (assay buffer) and finally re-suspended in 3 mL of the assay buffer and refrigerated until used in a detection assay.

### Example 4

### Detection of Target Molecule Using Molecule-Modified Nanoparticles

Materials CodeLink slides were obtained from GE Healthcare and printed with amino capture oligonucleotides using the manufacturer's recommended methods. Oligonucleotide capture probes and the control oligo were purchased from Integrated DNA Technologies and used without further purification. Barcode Capture sequence 5′TCT AAC TTG GCT TCA TTG CAC CGT T/3AmM-3′ (SEQ ID NO: 1) (where 3AmM is a amino modifier C6); Control Capture sequence 5′AAT GCT CAA TGG ATA CAT AGA CGA GG/3AmM/3′ (SEQ ID NO: 2) Barcode sequence: 3′-G-DTPA-T₁₉-ACC-GAA-GTA-ACG-TGG-CAA-T-Tosyl (SEQ ID NO: 3) Wash A, B, A₂₀ signal probe (SEQ ID NO: 4), hybridization chambers, the Shabbona research platform, and silver amplification solutions were purchased from Nanosphere Inc. and used according to manufacturer's recommended methods. Iodine solution (0.1N volumetric standard) was obtained from Aldrich Chemicals. PSA (90:10 WHO PSA standard; 90% bound: 10% free) was used as the standard for calibration curves throughout the study.

Hybridization. Microarrays (CodeLink slides; GE Healthcare) were printed at Nanosphere with bar code capture oligonucleotides (complementary to specific bar code sequence) and control sequences (noncomplementary sequence), whereby each slide received 10 arrays per slide with six repeats of each capture sequence per array. Nanosphere hybridization chambers were attached to each slide, separating each array physically. After loading 55 μL of bar codes, the slides were incubated for 60 min at 40° C. with shaking at 600 rpm. Signal probe mix (55 μL containing 50 μL of release buffer and 5 μL of 10 nM 15-nm dA₂₀ (SEQ ID NO: 4) gold nanoparticle probe (Nanosphere, Inc.)) was added, and incubation continued for 30 min. After hybridization, the slides were washed three times for 1 min in Wash A (0.5 N NaNO₃, 0.02% Tween 20, 0.01% SDS), then twice in Wash B (0.5 N NaNO₃) for 1 min. After a final quick wash (1-2 s) in 0.1N NaNO₃, the slides were spun dry.

A series of human serum samples were screened for the presence or absence of Prostate Specific Antigen (PSA) using the PSA antibody nanoparticles prepared in Example 3, via the bio-barcode assay (see, e.g., U.S. Pat. No. 6,495,324). A Shabbona liquid handling station equipped with a magnetic separation and agitation device was used when appropriate in this example. A sample block containing a series of calibration standards in serum and unknown samples were prepared by the addition of 30 μL of the assay buffer containing 1% polyacrylic acid sodium salt (15,000 MW) to the test wells. To this solution was added 30 μL of serum followed by 40 μL (1.5 μg per reaction well) of magnetic particles (MyOne Tosylated particles from Invitrogen) that had been previously functionalized using the manufacturer's recommended methods with PSA monoclonal Ab (Abcam Ab 403). The mixture was then agitated (1200 rpm) for one hour at room temperature and washed twice using the 1% assay buffer with concurrent magnetic separation. Gold nanoparticles (50 μL, 150 μM) were then delivered to the test wells and the mixture was then agitated for an hour at room temperature. To this mixture was added 150 μL of the assay buffer followed by magnetic separation. After five subsequent washes with 200 μL of the assay buffer, and the exchange of the assay buffer for the elution buffer (2×PBS, 0.04% Tween 20, 190 μL), the sample was transferred to PCR tubes and 10 μL of a 0.1 N iodine solution in water was added followed by 10 μL of a hybridization standard to a final concentration of 10 fM. This mixture was then heated at 95° C. for ten minutes to release the barcodes from the gold nanoparticles by dissolving the gold (Templeton, et al., J. Am. Chem. Soc., 120:1906 (1998); Kim, et al., J. Am. Chem. Soc., 122:7616 (2000); Puddephatt, The Chemistry of Gold, Elsevier, Amsterdam, 1978), allowed to cool to room temperature and transferred to hybridization chambers (Nanosphere Inc.) equipped with a CodeLink™ (GE Healthcare) glass slide that was previously printed with amino capture oligonucleotides complementary to the barcode and the control oligonucleotides. The assembly was then placed in an incubator for 60 minutes at 40° C. with agitation at 600 rpm. The glass slide was then washed twice with Wash A (Nanosphere Inc.) solution and reassembled with a new hybridization chamber. To each well of the assembly was added a hybridization solution containing fresh elution buffer and A₂₀ (SEQ ID NO: 4) signal probe (Nanosphere Inc.) at a final concentration of 1 nM. The entire assembly was placed in 40° C. incubator with agitation at 600 rpm for 30 minutes. The assembly was then disassembled and washed twice with Wash A. The slides were then washed three times with Wash B (Nanosphere Inc.) solution and spin dried. The silver development was carried out using Nanosphere Inc. silver amplification solution for five minutes at room temperature.

Bar Code Signal Detection. Equal volumes of Signal Enhancement A and Signal Enhancement B (both from Nanosphere, Inc.) were mixed and poured immediately over the slides inside plastic slide holders and incubated for 5 min at room temperature. Reactions were stopped with two washes in water and a thorough rinse in water. Slides were spun dry, and the back of slides were carefully wiped to remove dust, salt, and other contaminants. The slides were scanned on a Verigene™ ID system (Nanosphere).

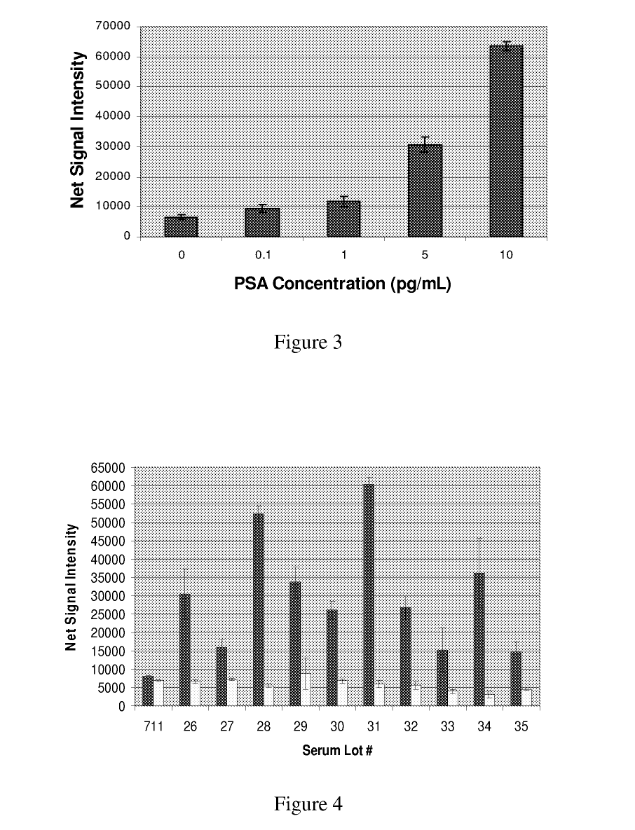

Bar Code Image Analysis. Scanned images (16-bit TIFF from Verigene ID) were analyzed with GenePix Pro v5.1 software (Axon Instruments). Mean spot intensities were first corrected for local background (mean pixel value of a similarly sized area in the vicinity of each spot) to generate raw spot intensities. FIG. 3 shows the calibration curve in assay buffer containing 1% PAA. The results shown in FIG. 3 demonstrate a representative sample run with multiples of calibration standards in assay buffer on the automated platform. Representative automation run using WHO PSA standard in assay buffer. The standards were prepared by spiking of known concentrations of the PSA antigen into the assay buffer at 0, 0.1 1.0, 5.0, and 10.0 pg/mL followed by the bio-barcode assay on the automated system and scanometric detection of the barcode DNA strands released from the 30 nm Au NP probes for PSA target titration. The gray scale images from Verigene ID system are converted into colored ones using GenePix Pro 6 software (Molecular Devices).

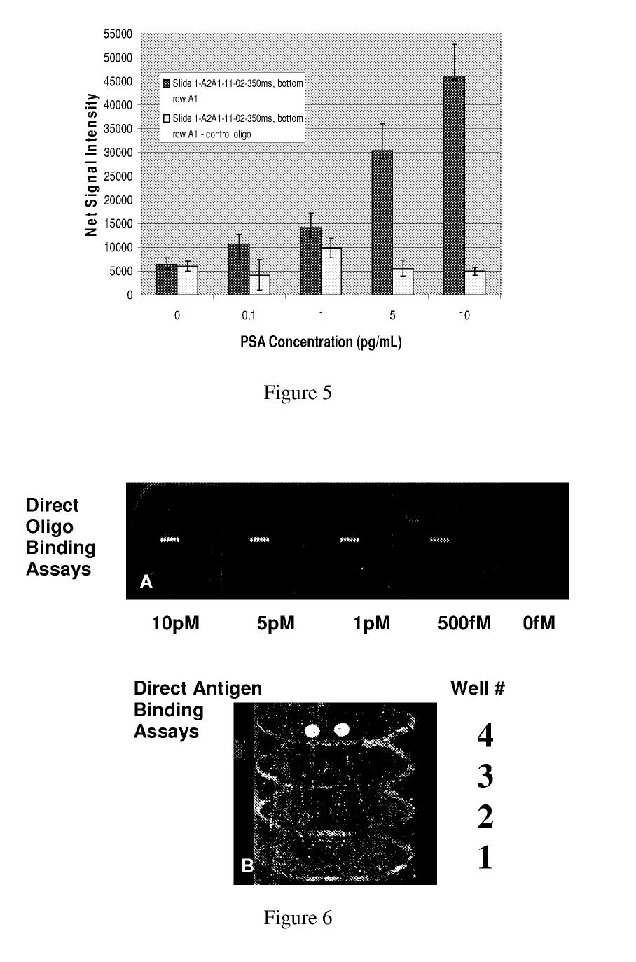

FIG. 4 shows the serum screening using 30% human serum containing 1% PAA. Representative automation run using 30% human serum. The samples were prepared by addition of human serum to the assay buffer on the automated system and scanometric detection of the barcode DNA strands released from the 30 nm Au NP probes for PSA target detection. The gray scale images from Verigene ID system are converted into colored ones using GenePix Pro 6 software (Molecular Devices). The 711 serum was determined to have the lowest PSA background and suitable for calibration curves in human serum. This serum was used to generate the calibration curves by spiking known amounts of PSA standard into the serum and carrying out the protein bio-barcode assay. The results shown in FIG. 5 demonstrate the PSA calibration curve in human serum obtained from the automated system.

FIG. 5 shows the calibration curve in 30% human serum containing 1% PAA. Representative automation calibration curve using 30% human serum. The samples were prepared by spiking 0.1, 1.0, 5.0, and 10.0 pg/mL of PSA standard into human serum followed by the addition of human serum to the assay buffer on the automated system and scanometric detection of the barcode DNA strands released from the 30 nm Au NP probes for PSA target detection.

Bio-Barcode Probe Characterization. Two separate methodologies were developed to demonstrate the dual nature of the bio-barcode probes. Since the probes are chimeric (both barcode and antibody are attached to the same nanoparticle), it was necessary to demonstrate the fidelity of the probe through a protein assay (to determine the activity of the antibody) and an oligonucleotide assay (to examine the activity of the oligonucleotide barcode). Such assays were devised by printing either the antigen or the oligonucleotide barcode capture sequences separately on the surface of CodeLink glass slides. After chemical coupling of these molecules, the printed surfaces were challenged with the bio-barcode probe in separate experiments. The dual binding and selectivity of these novel and sensitive probes could be successfully demonstrated in these assays. Panel A of FIG. 6 shows the results of an oligonucleotide hybridization assay using the bio-barcode probes, whereas Panel B demonstrates the antigen binding capability of the same bio-barcode probe in assay buffer. Direct binding assays of the bio-barcode probes via oligonucleotide hybridization and antibody antigen binding. Panel A of FIG. 6 shows the concentration dose/response of probe dilution series from 10 pM-OfM challenged with printed capture probes. Panel B of FIG. 6 shows results of the surface bound antigen challenged with bio-barcode probe (well 4), assay buffer (well 3), bio-barcode probe plus excess antigen (well 2), and bio-barcode probe plus excess antibody (well 1).

What is claimed:

1. A molecule-modified nanoparticle comprising a molecule covalently attached to an oligonucleotide, the oligonucleotide further covalently attached to a nanoparticle.

2. The molecule-modified nanoparticle of claim 1, wherein the oligonucleotide has a first end and a second end, and the molecule is attached at the first end and the nanoparticle is attached at the second end.

3. The molecule-modified nanoparticle of claim 1, wherein the molecule is a biomolecule.

4. The molecule-modified nanoparticle of claim 3, wherein the biomolecule is selected from the group consisting of a protein, a peptide, an antibody, a lipid, a carbohydrate, and combinations thereof.

5. The molecule-modified nanoparticle of claim 2, wherein the biomolecule is an antibody.

6. The molecule-modified nanoparticle of claim 1, wherein the nanoparticle has a diameter of about 10 nm to about 100 nm.

7. The molecule-modified nanoparticle of claim 1, wherein the nanoparticle is metallic.

8. The molecule-modified nanoparticle of claim 7, wherein the metal is selected from the group consisting of gold, silver, platinum, aluminum, palladium, copper, cobalt, indium, nickel, and mixtures thereof.

9. The molecule-modified nanoparticle of claim 1, wherein the nanoparticle comprises gold.

10. The molecule-modified nanoparticle of claim 9, wherein the oligonucleotide is attached to the nanoparticle via a functional group moiety, said functional group moiety comprising a sulfur atom.

11. The molecule-modified nanoparticle of claim 10, wherein the oligonucleotide was prepared using a dithiol phosphoramidite (DTPA).

12. The molecule-modified nanoparticle of claim 1, wherein the oligonucleotide is 20 nucleobases to 150 nucleobases in length.

13. A method of preparing a molecule-modified nanoparticle comprising
a) contacting a nanoparticle with an oligonucleotide having a functional group moiety toward a first end and a leaving group toward a second end to form an oligonucleotide-modified nanoparticle such that the oligonucleotide is attached to a surface of the nanoparticle via the functional group moiety; and
b) contacting the oligonucleotide-modified nanoparticle of (a) with a molecule having a nucleophile under conditions sufficient to permit displacement of the leaving group on the oligonucleotide by the nucleophile of the molecule to form the molecule-modified nanoparticle.

14. The method of claim 13, wherein the molecule is a biomolecule.

15. The method of claim 14, wherein the biomolecule is selected from the group consisting of a protein, a peptide, an antibody, a lipid, a carbohydrate, and combinations thereof.

16. The method of claim 15, wherein the biomolecule is an antibody.

17. The method of claim 13, wherein the nucleophile is a hydroxyl, amine, or thiol.

18. The method of claim 13, wherein the leaving group is selected from the group consisting of tosyl, mesyl, trityl, substituted trityl, nitrophenyl, chlorophenyl, fluorenylmethoxy carbonyl, and succinimidyl.

19. The method of claim 13, wherein the nanoparticle is metallic.

20. The method of claim 19, wherein the metal is selected from the group consisting of gold, silver, platinum, aluminum, palladium, copper, cobalt, indium, nickel, and mixtures thereof.

21. The method of claim 19, wherein the nanoparticle comprises gold.

22. The method of claim 13, wherein the nanoparticle has a diameter of about 10 nm to about 100 nm.

23. The method of claim 13, wherein the oligonucleotide is 20 nucleobases to 150 nucleobases in length.

24. A method of detecting an analyte in a sample comprising
a) contacting the sample with a molecule-modified nanoparticle of claim 1 under conditions to permit binding of the analyte to the molecule, and
b) detecting the analyte bound to the molecule-modified nanoparticle, wherein the binding of the analyte to the molecule-modified nanoparticle produces a detection event.

25. The method of claim 24, wherein the molecule is a biomolecule.

26. The method of claim 25, wherein the biomolecule is selected from the group consisting of a protein, a peptide, an antibody, a lipid, a carbohydrate, and combinations thereof.

27. The method of claim 24, wherein the oligonucleotide is at least partially complementary to a probe oligonucleotide and the oligonucleotide and probe oligonucleotide are hybridized.

28. The method of claim 27, wherein the detection event comprises melting the hybridized oligonucleotide and probe oligonucleotide and detecting the probe oligonucleotide.

29. The method of claim 24, wherein the detection is sensitive to detect the analyte at a concentration down to about 300 fM.


SEQUENCE LISTING (4 sequences)

SEQ ID NO: 1 - DNA, 25 bp, Artificial Sequence
  Synthetic oligonucleotide
tctaacttggcttcattgcaccgtt

SEQ ID NO: 2 - DNA, 26 bp, Artificial Sequence
  Synthetic oligonucleotide
aatgctcaatggatacatagacgagg

SEQ ID NO: 3 - DNA, 39 bp, Artificial sequence
  Synthetic oligonucleotide
taacggtgcaatgaagccatttttttttttttttttttg

SEQ ID NO: 4 - DNA, 20 bp, Artificial sequence
  Synthetic oligonucleotide
aaaaaaaaaaaaaaaaaaaa